(19) United States
(12) Patent Application Publication
(10) Pub. No.: US 2006/0177504 A1
(43) Pub. Date: Aug. 10, 2006

(54) Combination pain medication

(76) Inventor: Renjit Sundharadas, Milwaukee, WI (US)

Correspondence Address: Dr. Renjit Sundharadas, M.D., 11454 Raedene Way, San Diego, CA, 92131, US

(21) Appl. No.: 11/349,587

(22) Filed: Feb. 8, 2006

Related U.S. Application Data
(60) Provisional application No. 60/651,034, filed on Feb. 8, 2005.
(60) Provisional application No. 60/703,789, filed on Jul. 30, 2005.

(51) Int. Cl. A61K 31/60; A61K 31/405; A61K 31/192; A61K 31/4439

(52) U.S. Cl. 424/464; 514/338; 514/420; 514/569; 514/570; 514/165

(57) ABSTRACT

This patent is an evolution of previous combination medication patents. Previous combination patents such as U.S. Pat. No. 6,613,354 which is a combination of an NSAID and Proton Pump Inhibitor. Thus the previous patents have covered gastrointestinal prophylaxis but none has covered both gastrointestinal and cardiovascular prophylaxis. This is likely because the cardiovascular side effects of NSAIDs were only recently discovered. This patent thus represents a leap in safety in a class of medication that is used by millions of Americans on a daily basis. This combination would thus decrease morbidity and mortality.

### CROSS-REFERENCE TO RELATED APPLICATIONS

This application cross references 2 previous provisional patent applications filed on Feb 8, 2005 and Jul. 30, 2005. The USPTO numbers are 60/651034 and 60/703789. There are also the numbers 113009 and 113264 on each return postcard respectively.

This patent addresses the side effects of NSAID which are GI bleeds as well as cardiovascular complications. It does so by combining an NSAID along with Acid blockers and anti-coagulation medication. The acid blockers could be a proton pump inhibitor and/or a H2 blocker whereas the anticoagulant could be aspirin.

### STATEMENT REGARDING FEDERALLY SPONSORED RESEARCH OR DEVELOPMENT

None

### REFERENCE TO SEQUENCE LISTING, A TABLE, OR A COMPUTER PROGRAM LISTING COMPACT DISC APPENDIX

None

### BACKGROUND

A review of the current patent literature and medical literature shows various combination medication treatments for pain for the prescription and over the counter markets. Most recently questions have arisen regarding the safety of the Cox-2 class of drug. These concerns have mainly to do with these medications causing stroke, myocardial infarction and other cardiovascular side effects. Even Celebrex, which has not been pulled from the market, has had the consumer group, Public Citizen, call for this medication to be pulled from the market. Additionally, Naproxen, which has been used for many years as an over the counter medication has also been found to have cardiovascular side effects at increased doses. The safety of the class of medications called Non-steroidal anti-inflammatories (NSAIDs) as a whole could be called into question, as many long-term studies on these medications have not been done. One fact that seems to be consistent with all the studies performed is that increasing doses of NSAIDs over extended periods of time, such as 18 months, can cause increased side effects. However, the question thus remains as to what the side effects of lower doses over even longer terms could be since this would be very difficult to study. Thus, the patient population may develop fear of side effects and demand safer alternatives. This provisional patent application addresses these concerns by combining FDA approved medications to meet this need. A review of current and pending patents clearly shows that there have been combinations of medications that are already patented but have not made it to market in prescription or non-prescription form. This patent application addresses this need by exposing weaknesses in the current patent filings that are preventing these medications from reaching the market place. The new combination medication proposed would have various forms, some being prescription and others being non-prescription. Patient safety would thus be preserved and the medications would be safer than current over the counter medications, thus giving the consumer more options.

The main benefit of cox-2 inhibitors such as Celebrex is that they inhibit pain without causing gastrointestinal side effects as they inhibit prostaglandin production to lesser extent. The gastrointestinal side effects of some of the other NSAIDS can possibly be minimized or even avoided by including other appropriate prophylactic drug(s) in the formulation of the medication. Similarly, cardiovascular side effects also can be minimized or avoided by including appropriate prophylactic drugs. Such a combination product most likely would be safer and has the potential to capture market share and even earn a brand name capturing customer following. Several such combinations are proposed in the following sections of this application. Most if not all over the counter NSAIDs do not have ingredients to prevent gastric or cardiovascular side effects. Such a product most likely would take market share, as it would be safer. Also, there would be a tremendous opportunity to create a name brand since there are already many name brands that have a significant following from consumers.

### BRIEF SUMMARY OF THE INVENTION

This invention involves combining medications to reduce the side effects from NSAIDs which by definition also include Cox-2 inhibitors. These side effects include gastrointestinal side effects such as gastrointestinal bleeds as well as Cardiovascular side effects. The cardiovascular side effects include stroke and heart attack. Theses side effects are increased with higher doses of medication. This combination would combine gastrointestinal prophylaxis medication with a mild anticoagulant to decrease both risks. This is very important to the consumer since millions upon millions of persons will be taking these medications. This patent is also written in such a way that there can be multiple useful combinations. Also, the final product would have to be made in careful proportions of medication so that there is appropriate gastrointestinal and cardiovascular prophylaxis.

### BRIEF DESCRIPTION OF THE SEVERAL VIEWS OF THE DRAWINGS

No Drawings

### DETAILED DESCRIPTION OF THE INVENTION

This patent application aims to meet these needs in the following manners. First, to limit side effects this application it would combine an NSAID with an H2 blocker, Proton pump inhibitor (PPI), antacid, a prostaglandin, or a combination of the preceding. Currently there are PPI and H2 blocker combinations already patented, but these have not made it to market. A review of their patents shows that they add the minimum recommended dose for the medication to each tablet. However, this neglects how people actually use the medication, especially in the over the counter market; namely multiple tablets through a given period of time. Thus someone taking this type of medication could surpass the recommended daily maximum dose of a proton pump inhibitor or an H2 blocker. Since they would end up taking higher than normal doses, it thus makes sense as to why this type of medication is not widely available.

This patent application addresses this issue by claiming the practice of putting in sub therapeutic doses of any acid blocking agent so that the patient only gets significant acid production blockage if that patient's NSAID dose (or number of tablets) is high enough. This correlates with the dose of medication since higher doses cause increased gastric side effects. Thus, this patent also prevents current patent holders from bringing a product such as that to the market. Thus the product could be brought to market as an over the counter medication and stronger forms could also be brought to the market as a prescription form. Additionally, there is now no current patent that combines an NSAID with a proton pump inhibitor AND an H2 blocker. This is also important since these medications have different half lives and a sub therapeutic combination of these medications would provide short and long term coverage from gastric side effects that would more closely mirror the way patients use these medications. Also, since you are blocking more than one receptor related to acid production, at increasing doses there will be a synergistic effect giving much more acid production as the NSAID dose increases.

Finally, there's the question of stroke, myocardial infarction and other cardiovascular side effects. I propose adding a subtherapeutic or therapeutic dose of aspirin, ticlopidine, clopidogrel or any other cardiovascular prophylaxis agent to such a pill in therapeutic or sub therapeutic doses so that when the patient has taken a significant amount of medication, there is prophylaxis against cardiovascular side effects. Additionally, adding this third component can create a three-component pain medication consisting of a non-aspirin NSAID, an acid blocker (proton pump inhibitor, H2 blocker, etc.), and a medication such as aspirin to prevent cardiovascular side effects.

The series of claims that follows can represent a method of treating or prophylaxis of a condition in a human or mammal selected from the group consisting of angina, aorto-pulmonary shunt occlusion, colorectal cancer, esophageal cancer, colon cancer, coronary artery disease, dementia, dysmenorrhea, myocardial infarction, rheumatoid arthritis, osteoarthritis, pain, headache, migraine headache, stroke, thrombocythemia, post-operative thrombeoembolism, ischemia, bursitis, cognative decline, fever, gout, musculoskelatal disorders, soft tissue injury, pyrexia, asthma, bone resorption, cardiovascular diseases, atherosclerosis, pain, cancer, Alzheimer disease, cancer pain, and pericarditis; wherein the method comprises administering to a patient having one or more of the above conditions a composite medication as claimed in this provisional patent application.

There are many examples of how a medication could work in prescription form as well as over the counter. Any medication may be derived in different gastric prophylactic and cardiovascular prophylactic strengths with the lower strengths being available without a prescription and the higher strengths being available with a prescription only. The following is an over the counter version:

### EXAMPLE

An over the counter medication contains 200 mg Ibuprofen per tablet and is supposed to be taken as four tablets four times daily as the maximum dose. Such tablet may contain 1.25 mg of omeprazole per tablet so that when the 16 tablets are consumed, the total daily dose is 20 mg omeprazole per day, which is the starting daily dose. Additionally, this tablet could contain 2.5 mg of famotidine such that when 16 tablets are consumed it totals 40 mg of famotidine per day, which is the starting daily dose. Similarly, this medication could contain 5 mg of ASA meaning that after 16 pills, the patient would have cardiovascular prophylaxis of 80 mg per day. Depending on the product, there could be more or less of different components depending on whether or not this product is intended for prescription or non-prescription use, and depending on the components of the pill itself. As an example, any pill combined with Aspirin would promote GI bleeding and may need larger doses of gastrointestinal protective medications. Both Prescription and Over the counter medications could be divisible in order to help with dosing. Additionally this type of medication could be dispensed as a daily pack with each tablet having different levels of subcomponents to regulate intake of each subcomponent.

1. A combination of a, b, c, and d where a, b, c and d are the following:
a. one or more NSAIDs,
b. zero, one or more stroke/cardiovascular prophylaxis medications
c. zero, one or more gastric prophylaxis agent including antacids, prostaglandins or prostaglandin analogues
d. combination may or may not include acetominophen.

2. Any component or components in claim 1 may be sustained, immediate, or delayed release, quickly dissolving, delayed dissolving, sequentially dissolving, or have an enteric coating in order to alter the dissolving rate.

3. All Components in claim 1 may be in a dose range of 0.01-5000 mg per tablet.

4. The gastric prophylactic agent may includes proton pump inhibitors, H2 blockers and other agents such as antacids, protaglandins or prostaglandin analogues. The proton pump inhibitor is selected from but not limited to the group consisting of omeprazole, lansoprazole, rabeprazole, pantoprazole, leminoprazole, single enantiomers thereof, alkaline salts thereof and mixtures thereof. The H2 Blocker is selected from but not limited to the group consisting of cimetidine, nizatidine, ranitidine, roxatidine, famotidine, ebrotidine, burimamide, metiamide, tiotidine or oxmetidine, and pharmaceutically acceptable salts thereof. Any antacid used in combination with other medications in claims 1 may include, but not be limited to CaCO3, Mg(OH)2, Al(OH)3, combination of the aforementioned (i.e. more than one) or salt thereof.

5. The NSAID is selected from but not limited to the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and mixtures thereof. The NSAID is also defined as including the group of COX-2 inhibitors. A composition in which the COX-2 inhibitor is a compound or a pharmaceutically acceptable salt thereof, or any hydrate thereof selected from but not limited to rofecoxib, etoricoxib, celecoxib (Celebrex), valdecoxib, parecoxib, Vioxx, or a 5-alkyl-2-arylaminophenylacetic acid derivative COX-2 inhibitor, e.g. COX189. This group of NSAIDs is inclusive of COX-2 inhibitors known by trade name as Arcoxia, Dynastat, and Prexige.

6. The cardiovascular prophylaxis agent is selected from the group consisting of aspirin, clopidogrel, ticlopidine, aggrenox, dipyridamole, cilostazol, trental, or salt thereof. However, the cardiovascular prophylaxis agents may also include, HMG-CoA reductase inhibitors, ACE inhibitors, Angiotensin Receptor Blockers, Calcium Channel Blockers, and any other vasodilatory or anti-hypertensive agents or platelet aggregation inhibitors, and oral anticoagulants. The group of HMG-CoA reductase inhibitors, ACE inhibitors, Angiotensin Receptor Blockers, Calcium Channel Blockers and any other vasodilatory or anti hypertensive agents, platelet aggregation inhibitors or oral anticoagulants is to be selected from but not limited to the following list. Benazepril, captopril, enalapril, fosinopril, lisinopril, moexipril, perindopril, quinapril, ramipril, trandolapril, candesartan, eprosartan, ibesartan, losartan, telmisartan, valsartan, atorvastatin, cerivastatin, fluvastatin, lovastatin, pravastatin, simvastatin, aggrenox, dipyridamole, amlodipine, felodipine, isradipine, nicardipine, nifedipine, nisoldipine, diltiazem, verapamil, isosorbide dinitrate/mononitrate, combination of the aforementioned, or salt thereof.
